(19) United States
(12) Patent Application Publication
(10) Pub. No.: US 2023/0355186 A1
(43) Pub. Date: Nov. 9, 2023

(54) MOTION EVALUATION METHOD, COMPUTER PROGRAM, AND MOTION EVALUATION SYSTEM

(71) Applicant: NIPPON TELEGRAPH AND TELEPHONE CORPORATION, Tokyo (JP)

(72) Inventors: Kentaro TANAKA, Musashino-shi (JP); Shingo TSUKADA, Musashino-shi (JP); Masumi YAMAGUCHI, Musashino-shi (JP); Takayuki OGASAWARA, Musashino-shi (JP); Toichiro GOTO, Musashino-shi (JP)

(73) Assignee: NIPPON TELEGRAPH AND TELEPHONE CORPORATION, Tokyo (JP)

(21) Appl. No.: 18/021,849

(22) PCT Filed: Sep. 3, 2020
(86) PCT No.: PCT/JP2020/033449

§ 371 Date: Feb. 17, 2023

(51) Int. Cl. A61B 5/00; A61B 5/11

(52) U.S. Cl. CPC A61B 5/7275; A61B 5/11

(57) ABSTRACT

A motion evaluation method includes: a noise removal step of removing noise in time-series data related to a motion of a person; an extraction step of extracting data of an on-set section in which the motion of the person is performed from the time-series data from which the noise has been removed; a compression step of aligning a length of the extracted data of the on-set section for each on-set section and compressing the data of the on-set section through down-sampling processing; and an evaluation step of evaluating the motion of the person on the basis of the compressed data of the on-set section.

### TECHNICAL FIELD

The present invention relates to a motion evaluation method, a computer program, and a motion evaluation system.

### BACKGROUND ART

In the related art, a technique for classifying the posture and motion of a person by using time-series data related to the motion has been proposed. The time-series data related to the motion is data indicating the state of the body of a person acquired continuously in time. For example, the time-series data related to the motion is data obtained by continuously measuring the state of the body, such as surface myoelectric potential data, electrocardiographic data, and electroencephalogram data in biological information, or data obtained by measuring the external output of the body, such as acceleration data and pressure data.

In the technique for classifying the posture and motion of a person in the related art, for example, motion estimation such as classification of a difference between walking and running by using acceleration data or classification of a sitting position and a standing position by using acceleration data has been performed. Here, FIG. 9 shows an example of time-series data related to the motion. The time-series data related to the motion generally includes a section T1 including a waveform of the motion of the observation target and a section T2 not including a waveform of the motion of the observation target.

As a method of estimating the motion from time-series data, there is a method of using a Euclidean distance. In this method, the time information of the time-series data is maintained, partial time-series data indicating the motion to be analyzed is extracted from the time-series data, and the Euclidean distance between the pieces of extracted partial time-series data is obtained to estimate the motion. In this method, as shown in FIG. 10, first, partial time-series data indicating the motion to be analyzed is extracted from each piece of time-series data 1 and 2. Next, N (N is an integer of 1 or more) pieces of the partial time-series data extracted from the time-series data 1 are sampled to acquire an N-dimensional vector, and M (M is an integer of 1 or more) pieces of the partial time-series data extracted from the time-series data 2 are sampled to acquire an M-dimensional vector. In the method using the Euclidean distance, N = M is set. Then, the Euclidean distance between the acquired N-dimensional vector and M-dimensional vector is calculated.

However, determination of similarity of time-series data based on the Euclidean distance is weak against temporal distortion. That is, there is a weak point that the determination of similarity of time-series data based on the Euclidean distance is weak in classification between the case where the same motion is performed slowly and the case where the same motion is performed quickly.

A method such as dynamic time warping (DTW) in which a weak point of temporal distortion of the Euclidean distance is eliminated has also been proposed. In the DTW method, as shown in FIG. 11, first, partial time-series data indicating the motion to be analyzed is extracted from each piece of time-series data 3 and 4. Next, N pieces of the partial time-series data extracted from the time-series data 3 are sampled to acquire an N-dimensional vector, and M pieces of the partial time-series data extracted from the time-series data 4 are sampled to acquire an M-dimensional vector. In the method using DTW, it is not necessary that N = M, and N ≠ M may be used. Then, the distance between the acquired N-dimensional vector and M-dimensional vector is calculated by DTW.

### CITATION LIST

### Non Patent Literature

[NPL 1] Preece, Stephen J., John Y. Goulermas, Laurence P. J. Kenney, Dave Howard, Kenneth Meijer, and Robin Crompton. 2009. “Activity Identification Using Body-Mounted Sensors--a Review of Classification Techniques.” Physiological Measurement 30 (4) :R1-33.
[NPL 2] Berndt D, Clifford J (1994) Using dynamic time warping to find patterns in time series. AAAI-94 workshop on knowledge discovery in databases, pp 229-248

### SUMMARY OF INVENTION

### Technical Problem

In the case of the above-described method in the related art, a distance between data is calculated for each of a plurality of pieces of time-series data showing one motion, and the time-series data is classified by using the distance as a feature amount. Therefore, there is a disadvantage that a delicate difference in the interlocking between a plurality of pieces of time-series data indicating one motion is missing as information when calculating the distance between the data, and cannot be expressed. As a result, it is not suitable for evaluating the difference between good and bad for the same motion, for example.

In order to evaluate good or bad of such motion, it is necessary to take into account the difference in the interlocking timing of a plurality of muscles and the difference in the time timing of contraction for each of the plurality of muscles. However, in the method in the related art, these differences are lost due to the scale of the time-series data. Further, there is a problem that the evaluation accuracy is lowered because the section including only the noise other than the target signal is equally handled.

In view of the above circumstances, an object of the present invention is to provide a technique capable of improving the evaluation accuracy of the motion of a person.

### Solution to Problem

According to an aspect of the present invention, there is provided a motion evaluation method including: a noise removal step of removing noise in time-series data related to a motion of a person; an extraction step of extracting data of an on-set section in which the motion of the person is performed from the time-series data from which the noise has been removed; a compression step of aligning a length of the extracted data of the on-set section for each on-set section and compressing the data of the on-set section through down-sampling processing; and an evaluation step of evaluating the motion of the person on the basis of the compressed data of the on-set section.

According to another aspect of the present invention, there is provided a computer program causing a computer to execute: a noise removal step of removing noise in time-series data related to a motion of a person; an extraction step of extracting data of an on-set section in which the motion of the person is performed from the time-series data from which the noise has been removed; a compression step of aligning a length of the extracted data of the on-set section for each on-set section and compressing the data of the on-set section through down-sampling processing; and an evaluation step of evaluating the motion of the person on the basis of the compressed data of the on-set section.

According to still another aspect of the present invention, there is provided a motion evaluation system including: a sensor configured to acquire time-series data related to a motion of a person; a noise removal unit configured to remove noise in the time-series data; an extraction unit configured to extract data of an on-set section in which the motion of the person is performed from the time-series data from which the noise has been removed; a compression unit configured to align a length of the extracted data of the on-set section for each on-set section and compress the data of the on-set section through down-sampling processing; and an evaluation unit configured to evaluate the motion of the person on the basis of the compressed data of the on-set section.

### Advantageous Effects of Invention

According to the present invention, it is possible to improve the evaluation accuracy of the motion of a person.

### BRIEF DESCRIPTION OF DRAWINGS

FIG. 1 is a configuration diagram of a motion evaluation system according to the present invention.

FIG. 2 is a diagram showing features between time-series data desired to be captured in the present invention.

FIG. 3 is a block diagram showing a specific example of a functional configuration of a motion evaluation device according to an embodiment.

FIG. 4 is a block diagram showing a specific example of a functional configuration of a learning device according to the present embodiment.

FIG. 5 is a flowchart showing a flow of motion evaluation processing performed by the motion evaluation device according to the embodiment.

FIG. 6 is a diagram for describing a part of the motion evaluation processing performed by the motion evaluation device according to the present embodiment.

FIG. 7 is a schematic diagram showing a flow of processing for learning training data (learning processing) and processing for estimating an evaluation score on the basis of a trained model (estimation processing) in the present embodiment.

FIG. 8 is a diagram showing an example of a main use case of the present invention.

FIG. 9 is a diagram showing an example of time-series data related to a motion.

FIG. 10 is a diagram for describing a method of performing motion estimation from time-series data using a Euclidean distance.

FIG. 11 is a diagram for describing a method of performing motion estimation from time-series data using DTW.

### DESCRIPTION OF EMBODIMENTS

An embodiment of the present invention will be described below with reference to the drawings.

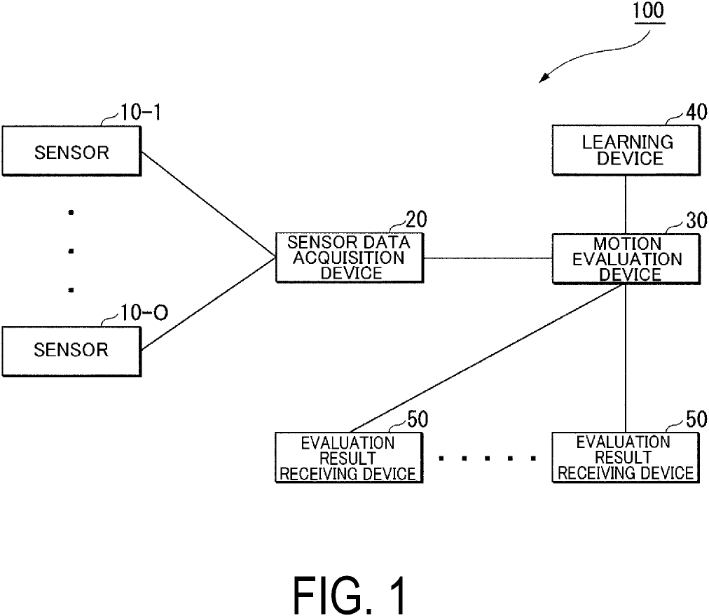

FIG. 1 is a configuration diagram of a motion evaluation system 100 according to the present invention. The motion evaluation system 100 includes one or more sensors 10-1 to 10-O (O is an integer of 1 or more), a sensor data acquisition device 20, a motion evaluation device 30, a learning device 40, and one or more evaluation result receiving devices 50.

The sensor 10 acquires biological information of a person (for example, surface myoelectric potential data, electrocardiographic data, and electroencephalogram data) in time series. The sensor 10 may measure the external output of the body, such as acceleration data and pressure data. The sensor 10 may be a wristband type sensor attachable to a person, or may be installed in a place where biological information, acceleration data, pressure data, and the like can be acquired from the person.

In the following description, a case where the sensor 10 acquires surface myoelectric potential data will be described as an example. The sensor 10 transmits the acquired surface myoelectric potential data to the sensor data acquisition device 20. The sensor 10 may transmit the acquired surface myoelectric potential data to the sensor data acquisition device 20 each time the acquired surface myoelectric potential data is acquired, or may collectively transmit the acquired surface myoelectric potential data to the sensor data acquisition device 20 for a certain period of time.

The sensor data acquisition device 20 acquires surface myoelectric potential data transmitted from the sensor 10, and manages the acquired surface myoelectric potential data for each sensor 10. In this way, the sensor data acquisition device 20 holds time-series data of the surface myoelectric potential data for each sensor 10. The sensor data acquisition device 20 transmits the held time-series data of the surface myoelectric potential data (hereinafter simply referred to as “time-series data”) to the motion evaluation device 30. The sensor data acquisition device 20 may transmit the held time-series data to the motion evaluation device 30 at a predetermined timing, or may transmit the time-series data requested from the motion evaluation device 30 to the motion evaluation device 30. The predetermined timing may be a preset time or a timing when a certain period of time has elapsed.

The motion evaluation device 30 evaluates the motion of the person by using the time-series data transmitted from the sensor data acquisition device 20. Evaluating the motion of the person means, for example, classifying the motion of the person into good or bad, and expressing the motion of the person numerically (hereinafter referred to as “scoring”). Hereinafter, the good or bad and the scoring of the motion of the person will be collectively described as an evaluation score. The motion evaluation device 30 evaluates the motion of the person by inputting time-series data to a trained model generated by, for example, the learning device 40. The motion evaluation device 30 is configured by using an information processing device such as a server, a laptop computer, a smartphone, and a tablet terminal.

The learning device 40 generates a trained model by training a learning model with the training data as input. The training data is data for training used for supervised learning, and is data represented by a combination of input data and output data assumed to have a correlation with the input data. The training data input to the learning device 40 is data in which a feature amount obtained on the basis of the time-series data is associated with an evaluation score. The feature amount obtained on the basis of the time-series data is generated by processing performed by the motion evaluation device 30 to be described later.

The learning device 40 inputs the time-series data and generates a trained model trained to output an evaluation score. Here, training (learning) is to optimize coefficients used in a machine learning model. For example, training (learning) is to adjust coefficients used in a machine learning model so that a loss function becomes a minimum. The coefficients used in the machine learning model are, for example, weight values and bias values.

The evaluation result receiving device 50 is a device that receives an evaluation result obtained by the motion evaluation device 30. For example, the evaluation result receiving device 50 is a device held by a person who is an evaluation target of a motion or a person related to the person. The evaluation result receiving device 50 is configured by using an information processing device such as a personal computer, a laptop computer, a smartphone, and a tablet terminal.

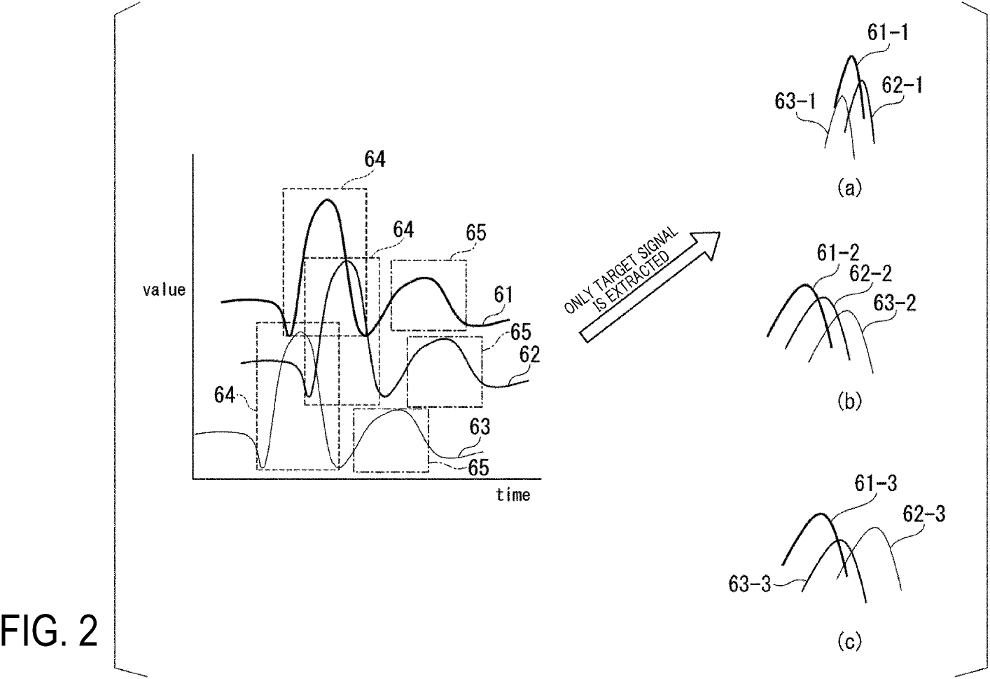

FIG. 2 is a diagram showing features between time-series data desired to be captured in the present invention.

A plurality of pieces of time-series data 61 to 63 are shown on the left side of FIG. 2. The time-series data 61 represents the time-series data of the surface myoelectric potential data obtained by the sensor 10-1. The time-series data 62 represents the time-series data of the surface myoelectric potential data obtained by the sensor 10-2. The time-series data 63 represents the time-series data of the surface myoelectric potential data obtained by the sensor 10-3. In these pieces of time-series data 61 to 63, a waveform of a target signal which is essentially desired to be captured (for example, a waveform representing a motion of a person), and noise are mixed. In the case of a non-stationary signal related to the motion of a person, there are a section including the waveform of the motion of the observation target and a section not including the waveform of the motion of the observation target. Therefore, in performing analysis, an analysis technique is required for only the waveform of the target signal.

In FIG. 2, waveforms surrounded by rectangles 64 are waveforms including the target signal and noise, and waveforms surrounded by rectangles 65 are waveforms including only noise. In the present invention, only the waveform of the target signal is extracted by extracting the waveform surrounded by the rectangle 64 from each piece of the time-series data 61 to 63 and removing noise. FIG. 2(a) shows waveforms 61-1, 62-1, and 63-1 after noise is removed from the waveform surrounded by the rectangle 64. The waveform 61-1 represents a waveform after noise is removed from the waveform surrounded by the rectangle 64 in the time-series data 61. The waveform 61-2 represents a waveform after noise is removed from the waveform surrounded by the rectangle 64 in the time-series data 62. The waveform 61-3 represents a waveform after noise is removed from the waveform surrounded by the rectangle 64 in the time-series data 63.

The waveforms 61-2, 62-2, and 63-2 and 61-3, 62-3, and 63-3 shown in FIGS. 2(b) and 2(c) are waveforms at a time different from the waveforms 61-1, 62-1, and 63-1 shown in FIG. 2(a). However, the waveforms shown in FIGS. 2(a) and 2(c) are waveforms obtained by extracting only the target signal from the waveforms including the target signal and the noise in the same time-series data 61, 62, and 63. In the present invention, the difference in interlocking between the sensors 10-1 to 10-3 is thus used as a feature amount.

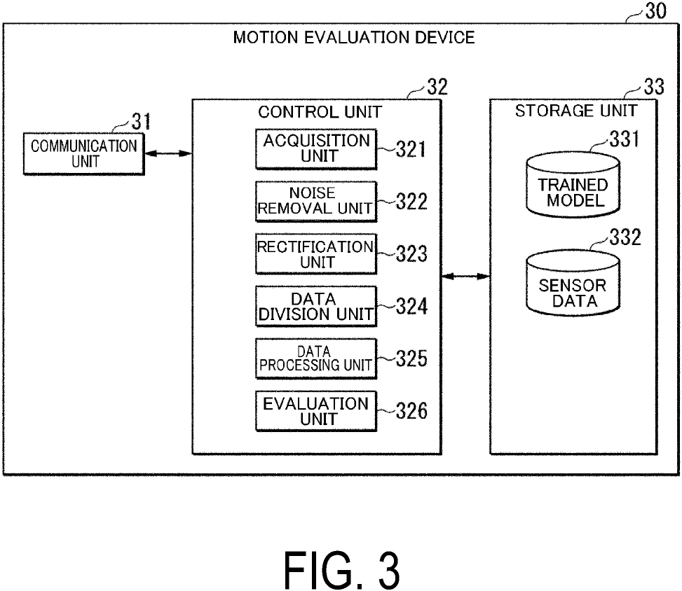

FIG. 3 is a block diagram showing a specific example of a functional configuration of the motion evaluation device 30 according to the present embodiment.

The motion evaluation device 30 includes a communication unit 31, a control unit 32, and a storage unit 33.

The communication unit 31 communicates with other devices. The other devices are, for example, the sensor data acquisition device 20 and the evaluation result receiving device 50. The communication unit 31 receives, for example, time-series data transmitted from the sensor data acquisition device 20. The communication unit 31 receives, for example, a trained model transmitted from the learning device 40. The communication unit 31 transmits an evaluation result to the evaluation result receiving device 50. When a trained model is recorded in an external recording medium such as a Universal Serial Bus (USB) memory or an SD card, the communication unit 31 receives the trained model via the external recording medium.

A trained model 331 and sensor data 332 are stored in the storage unit 33. The storage unit 33 is configured by using a storage device such as a magnetic storage device or a semiconductor storage device.

The trained model 331 is a trained model trained by the learning device 40. The trained model is associated with information on a coefficient optimized by the learning device 40.

The sensor data 332 is time-series data for each sensor 10 obtained from the sensor data acquisition device 20.

The control unit 32 controls the entire motion evaluation device 30. The control unit 32 is configured by using, for example, a processor such as a central processing unit (CPU) and a memory. The control unit 32 executes a program to realize functions of an acquisition unit 321, a noise removal unit 322, a rectification unit 323, a data division unit 324, a data processing unit 325, and an evaluation unit 326.

Some or all of the functional units of the acquisition unit 321, the noise removal unit 322, the rectification unit 323, the data division unit 324, the data processing unit 325, and the evaluation unit 326 may be realized using hardware (circuit part; including circuitry) such as an application specific integrated circuit (ASIC), a programmable logic device (PLD), or a field programmable gate array (FPGA) or may be realized by cooperation of software and hardware. The program may be recorded on a computer-readable recording medium. The computer-readable recording medium is, for example, a non-transitory storage medium such as a portable medium such as a flexible disk, a magneto-optical disk, a read only memory (ROM), or a CD-ROM, or a storage device such as a hard disk built in a computer system. The program may be transmitted via an electrical communication line.

Some of the functions of the acquisition unit 321, the noise removal unit 322, the rectification unit 323, the data division unit 324, the data processing unit 325, and the evaluation unit 326 do not need to be mounted on the motion evaluation device 30 in advance, and may be realized by installing an additional application program on the motion evaluation device 30.

The acquisition unit 321 acquires various types of information. The acquisition unit 321 acquires, for example, time-series data from the sensor data acquisition device 20. The acquisition unit 321 acquires, for example, a trained model from the learning device 40. The acquisition unit 321 may dynamically acquire various types of information or passively acquire it. Dynamically acquiring means that the acquisition unit 321 acquires information by requesting the information from the target device. Passively acquiring means that the acquisition unit 321 acquires information without requesting the information from the target device.

The noise removal unit 322 performs noise removal processing on the time-series data to be processed. When a general biological signal is targeted, the noise removal unit 322 performs processing such as band-pass filter processing or Wiener filter processing. The time-series data to be processed is, for example, time-series data of the sensor 10 attached to a designated person.

When the time-series data is signal data, a rectification unit 323 performs rectification processing on the time-series data subjected to noise removal processing. As the rectification processing, a method of obtaining the absolute value of data, a method of a root mean square value, and the like can be used, but the rectification processing is not particularly specified in the present invention.

The data division unit 324 estimates at least an on-set section in the time-series data to which noise removal processing has been performed, and divides the time-series data for each estimated on-set section. The on-set section is a section from a point where the person is assumed to have started the motion (hereinafter referred to as a “start point”) to a point where the person is assumed to have ended the motion (hereinafter referred to as an “end point”) on the time-series data. The start point is a point in time at which a value of the time-series data is compared with an average value of samples in an immediately preceding section and the value changes by a threshold value or more, that is, a point where a value of the time-series data is compared with a value in a certain section immediately before and the value increases or decreases by a threshold value or more. The end point is a point that approaches the average value again at a time after the start point.

The data division unit 324 estimates a section satisfying the condition in the time-series data as an on-set section. The data division unit 324 extracts the data of the estimated offset section from the time-series data. The on-set sections of the plurality of pieces of time-series data extracted in this way are compared, and the on-set sections overlapped with each other or included in a certain time before and after are defined as one data group.

The data processing unit 325 processes the data of the on-set section extracted by the data division unit 324. Specifically, the length of the data group of the on-set sections extracted by the data division unit 324 is irregular for each on-set section. Then, the data processing unit 325 performs processing for matching the start points of all the on-set sections with data having the earliest time at the start point and matching the end points of all the on-set sections with data having the latest time at the end point while maintaining the time-series information.

When the start point and the end point are matched, the data processing unit 325 fills all data corresponding to the outside of the on-set section with a value of 0 or puts a fixed value therein. Further, the data processing unit 325 performs down-sampling processing for making the length between data groups constant for the data groups in which the data lengths in the data groups are unified, and compresses the dimension of the data while holding the outline of the data.

When the data processing unit 325 performs the above processing, time distortion which is a weak point of similarity calculation due to Euclidean distance of the time-series data can be eliminated while leaving features of the waveform shape of data consisting of sequence between the time-series data and continuity of the on-set section, and a condition for fixing the number of samples required for calculation can be satisfied. For example, when the data processing unit 325 performs processing on data in which the on-set sections overlap and data in which the on-set sections hardly overlap, the time per sample of the latter is shorter than the time per sample of the former. The feature amount can be calculated by taking into account the interlocking in the data group which cannot be seen only by the distance between the respective data groups.

The evaluation unit 326 evaluates the motion of the person on the basis of the data group processed by the data processing unit 325. The evaluation unit 326 evaluates the motion of the person by inputting the processed data group to the trained model, for example. The evaluation unit 326 evaluates the motion of the person by calculating the distance between the processed data groups, for example.

When the motion evaluation device 30 is any one of a laptop computer a smartphone, and a tablet terminal, the motion evaluation device 30 is configured to include an input unit and a display unit.

The display unit is an image display device such as a liquid crystal display, an organic electro luminescence (EL) display, and a cathode ray tube (CRT) display. The display unit displays the evaluation result according to the operation of the user. The display unit may be an interface for connecting the image display device to the motion evaluation device 30. In this case, the display unit generates a video signal for displaying the evaluation result, and outputs the video signal to the image display device connected to the display unit itself.

An operation unit is configured by using existing input devices such as a keyboard, a pointing device (mouse, tablet, etc.), a touch panel, and buttons. The operation unit is operated by a user when inputting an instruction of the user to the motion evaluation device 30. For example, the operation unit receives an input of an evaluation start instruction of the motion of the person. Further, the operation unit may be an interface for connecting the input device to the motion evaluation device 30. In this case, the operation unit inputs an input signal generated in the input device in response to an input by the user to the motion evaluation device 30.

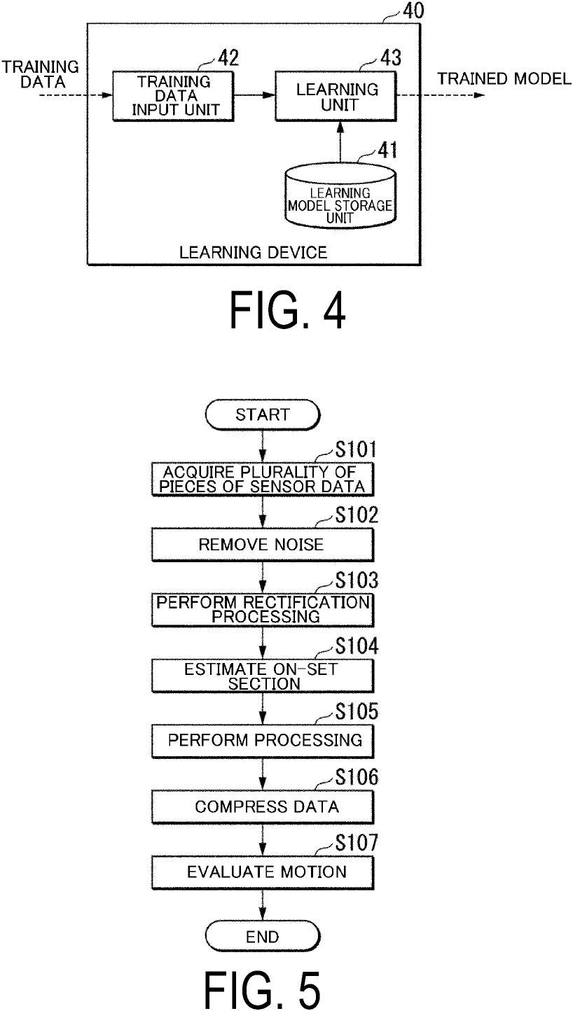

FIG. 4 is a block diagram showing a specific example of a functional configuration of the learning device 40 according to the present embodiment.

The learning device 40 includes a CPU, a memory, an auxiliary storage device, and the like, which are connected by a bus, and executes a program. The learning device 40 functions as a device including a learning model storage unit 41, a training data input unit 42, and a learning unit 43 by executing a program. Note that all or some of the functions of the learning device 40 may also be realized by using hardware such as the ASIC, the PLD, or the FPGA. The program may be recorded on a computer-readable recording medium. The computer-readable recording medium is, for example, a portable medium such as a flexible disk, a magneto-optical disk, a ROM, or a CD-ROM, or a storage device such as a hard disk built in a computer system. The program may be transmitted via an electrical communication line.

The learning model storage unit 41 is configured by using a storage device such as a magnetic storage device or a semiconductor storage device. The learning model storage unit 41 stores in advance a learning model in machine learning. Here, the learning model is information indicating a machine learning algorithm used in learning a relationship between input data and output data. Learning algorithms for supervised learning include various algorithms such as various regression analysis methods, decision trees, k-nearest neighbor algorithms, neural networks, support vector machines, deep learning, and the like, but any learning model may be used. In the present embodiment, a case where a neural network such as a multilayer perceptron is used as a learning model for machine learning will be described as an example.

The training data input unit 42 has a function of inputting training data. In the present embodiment, the feature amount obtained on the basis of the time-series data is used as input data, and the evaluation score corresponding to the input feature amount is used as output data. Here, the feature amount input to the training data input unit 42 is information on the evaluation score and data generated by the data processing unit 325. A combination of the input data and the output data is defined as one sample data, and a set of a plurality of pieces of sample data is generated in advance as training data.

For example, the training data input unit 42 may be communicably connected to an external device (not shown) that stores the training data generated in this way, and the training data may be input from the external device via the communication interface, or may be input to the learning device 40 after being generated by the motion evaluation device 30. Further, for example, the training data input unit 42 may be configured to input training data by reading the training data from a recording medium that stores the training data in advance. The training data input unit 42 outputs the training data thus input to the learning unit 43.

The learning unit 43 generates a trained model by learning training data output from the training data input unit 42 on the basis of a learning model. The generated trained model is input to the motion evaluation device 30. The input of the trained model to the motion evaluation device 30 may be performed via communication between the learning device 40 and the motion evaluation device 30, or via a recording medium in which the trained model is recorded.

Next, specific learning processing of the learning unit 43 will be described. First, the learning unit 43 calculates an error between an evaluation score obtained by inputting training data to a learning model and an evaluation score included in the training data. Then, the learning unit 43 solves a minimization problem about an objective function determined on the basis of the calculated error to update the coefficients used in the learning model. The learning unit 43 repeats updating of the coefficients until the coefficient used in the learning model is optimized or by a predetermined number of times. The coefficients of the learning model are estimated by an error backpropagation method and a stochastic gradient descent (SGD) method. As the optimization method, an optimization algorithm other than the stochastic gradient descent method may be used as long as the combination of the error backpropagation method and the following optimization algorithm is used. Optimization algorithms other than the stochastic gradient descent method include, for example, Adam, Adamax, Adagrad, RMSProp, Adadelta, and the like.

The learning unit 43 outputs the coefficient obtained by the above processing and the learning model to the motion evaluation device 30 as a trained model.

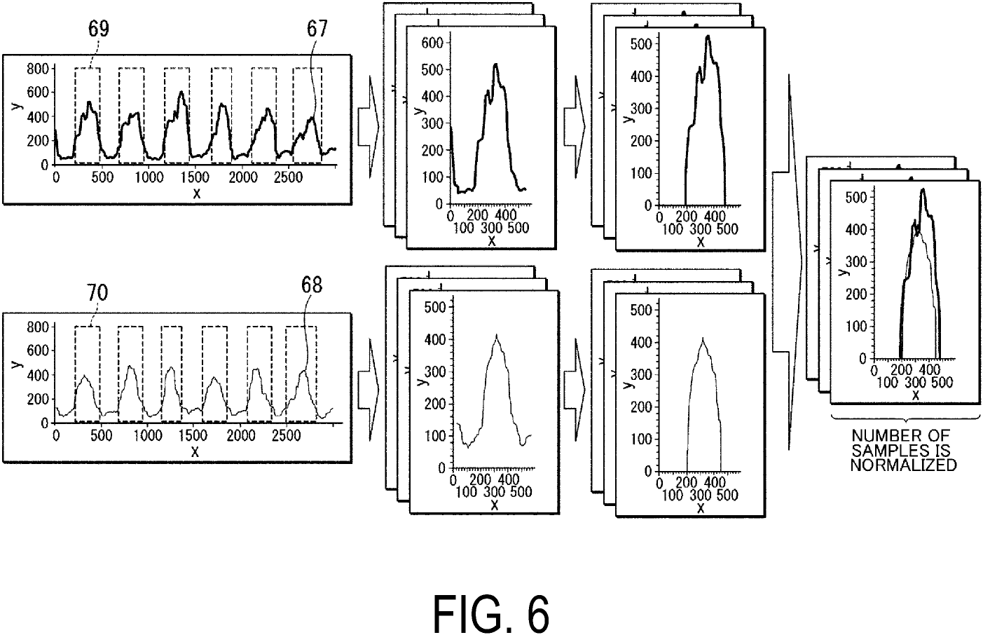

FIG. 5 is a flowchart showing a flow of motion evaluation processing performed by the motion evaluation device 30 according to the embodiment. The motion evaluation processing performed by the motion evaluation device 30 until processing data is generated will be described with reference to FIG. 6. FIG. 6 is a diagram for describing a part of the motion evaluation processing performed by the motion evaluation device 30 according to the present embodiment.

The acquisition unit 321 acquires a plurality of pieces of time-series data from the sensor data acquisition device 20 (step S101). The acquisition unit 321 records, for example, the time-series data acquired by the sensor 10-1 and the time-series data acquired by the sensor 10-2 in the storage unit 33 as the sensor data 332.

The noise removal unit 322 performs noise removal processing on each of the two pieces of time-series data recorded in the storage unit 33 as the sensor data 332 (step S102). The noise removal unit 322 outputs each piece of time-series data after the noise removal processing to the rectification unit 323. The rectification unit 323 performs rectification processing on each piece of time-series data after the noise removal processing (step S103). FIG. 6 shows an example in which the rectification unit 323 performs a root mean square on each piece of time-series data after the noise removal processing.

The time-series data 67 and 68 shown in FIG. 6 are time-series data obtained by the root mean square. The time-series data 67 corresponds to the time-series data obtained by the sensor 10-1, and the time-series data 68 corresponds to the time-series data obtained by the sensor 10-2.

The data division unit 324 estimates an on-set section of each piece of the time-series data 67 and 68 (step S104). In FIG. 6, an on-set section in the time-series data 67 is shown by a rectangle 69, and an on-set section in the time-series data 68 is shown by a rectangle 70. The data division unit 324 extracts the data of the estimated offset section from the time-series data. Then, the data division unit 324 compares on-set sections of the plurality of pieces of extracted time-series data with each other, and defines the on-set sections overlapped with each other or included in a certain time before and after as one data group. This corresponds to the second state from the left in FIG. 6.

Specifically, the data processing unit 325 performs processing on the data group of the on-set sections extracted by the data division unit 324 (step S105). Specifically, first, the data processing unit 325 performs processing for aligning the offset sections for each data group of the respective on-set sections. At this time, the data processing unit 325 fills all data corresponding to the outside of the on-set section with a value of 0 or puts a fixed value therein. Thus, the data length in the data group of the respective on-set sections is unified. This corresponds to the third state from the left in FIG. 6.

The data processing unit 325 combines the data groups of the respective on-set sections. Here, the combination means that the data groups of the respective on-set sections are superimposed. For example, the data processing unit 325 combines the data of the on-set section extracted from the time-series data 67 and the data of the on-set section extracted from the time-series data 68 on the same time axis.

In the example shown in FIG. 6, the number of the data of the on-set section extracted from the time-series data 67 is six, and the number of the data of the on-set section extracted from the time-series data 68 is six. Then, the data processing unit 325 combines the first data of the on-set section extracted from the time-series data 67 and the first data of the on-set section extracted from the time-series data 68. Similarly, the data processing unit 325 combines the data groups of the respective on-set sections. Thus, the combined six data groups are generated.

The data processing unit 325 performs down-sampling processing for normalizing the number of samples in each of the six data groups and making the length between the data groups constant, and thereby compresses the dimension of the data while holding the outline of the data (step S106). This corresponds to the fourth state from the left in FIG. 6. The data group is a feature amount used for learning processing in the learning device 40, and is data used for evaluation of a motion. The data processing unit 325 outputs the compressed data group to the evaluation unit 326 when only the time-series data is input to the motion evaluation device 30, and outputs the compressed data group to the learning device 40 as training data when the time-series data and the information on the evaluation score are input.

The evaluation unit 326 evaluates the motion by using the data group compressed by the data processing unit 325 (step S107). Specifically, the evaluation unit 326 acquires the evaluation score by inputting the data group compressed by the data processing unit 325 to the trained model 331. The evaluation unit 326 transmits the information on the acquired evaluation score to the evaluation result receiving device 50 via the communication unit 31.

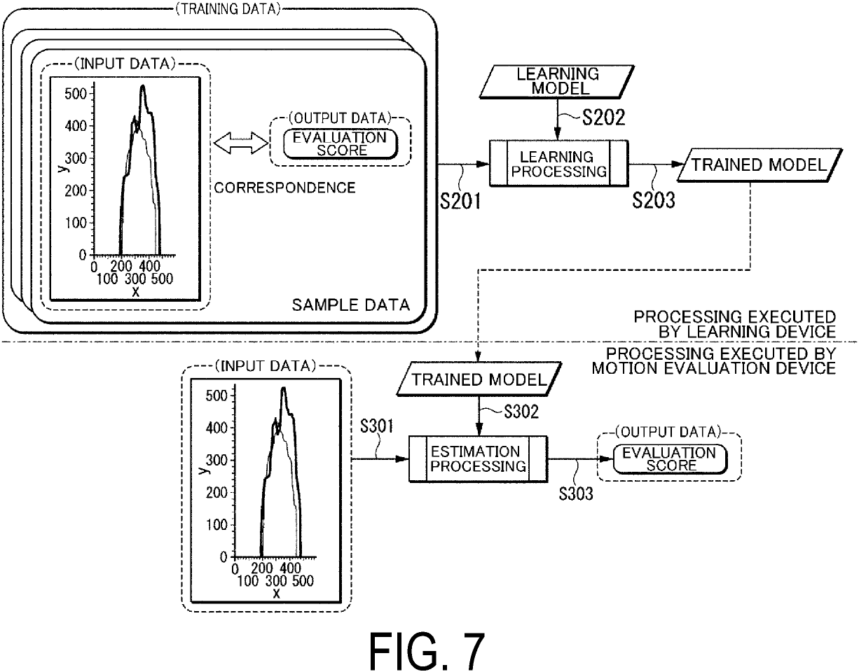

FIG. 7 is a schematic diagram showing a flow of processing for learning training data (learning processing) and processing for estimating an evaluation score on the basis of a trained model (estimation processing) in the present embodiment. First, in the learning device 40, the training data input unit 42 inputs training data, and outputs the input training data to the learning unit 43 (step S201). Subsequently, the learning unit 43 acquires a learning model from the learning model storage unit 41 (step S202). Subsequently, the learning unit 43 generates a trained model by executing learning processing of training data based on the learning model (step S203). The trained model thus generated is recorded in the storage unit 33 of the motion evaluation device 30.

On the other hand, first, the motion evaluation device 30 outputs the compressed data group obtained by processing from step S101 to step S106 shown in FIG. 5 to the evaluation unit 326 (step S301). Subsequently, the evaluation unit 326 acquires the trained model 331 from the storage unit 33 (step S302). Subsequently, the evaluation unit 326 inputs the acquired compressed data group to the trained model 331, and executes estimation processing for acquiring an evaluation score as an output thereof (step S303). The motion evaluation device 30 can estimate the evaluation score in time series by repeatedly executing the processing of step S301 to step S303.

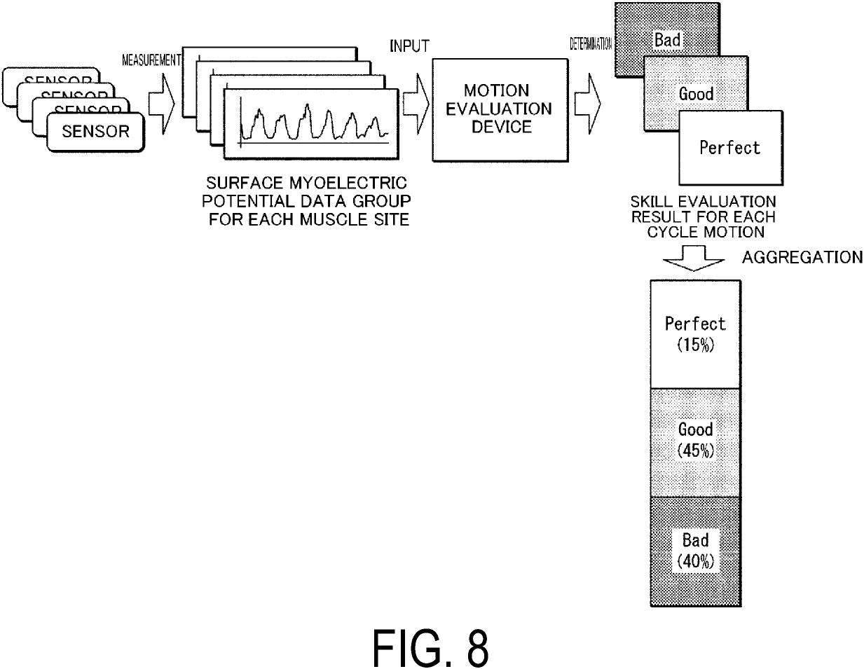

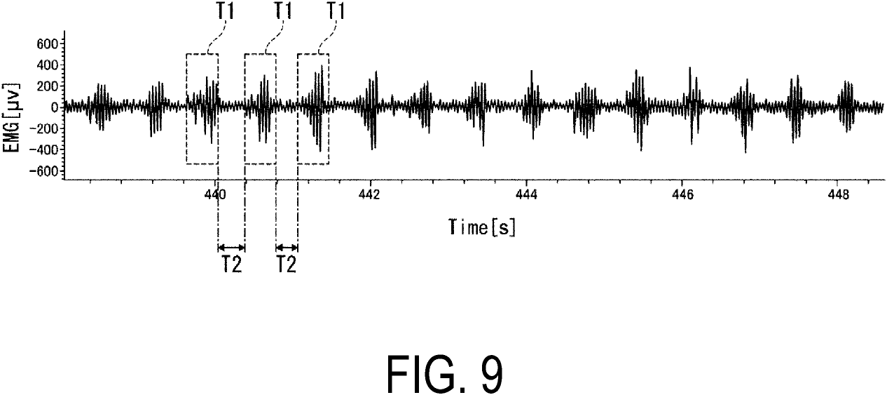

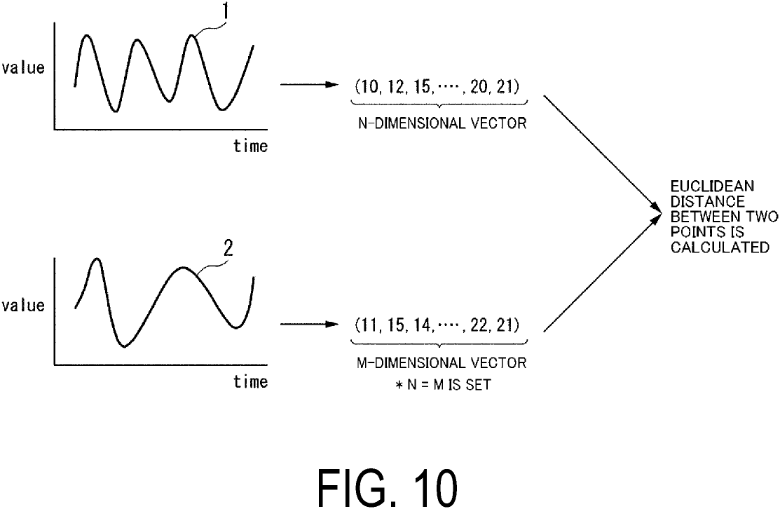

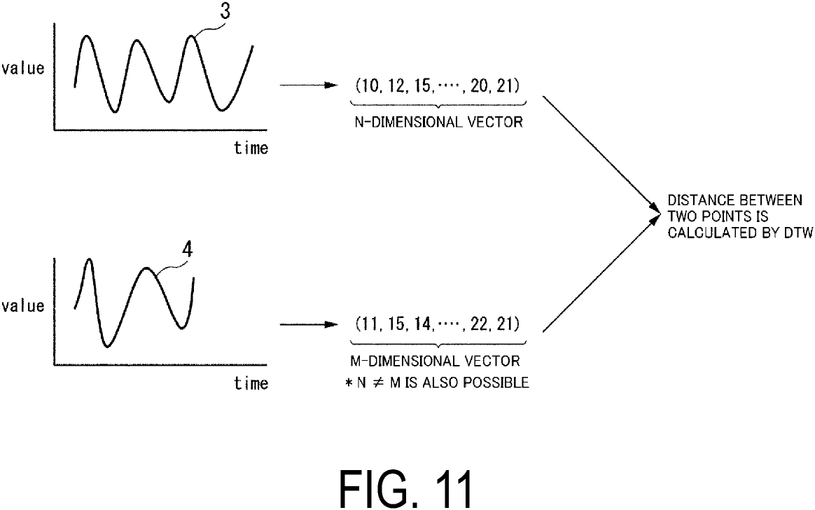

FIG. 8 is a diagram showing an example of a main use case of the present invention.

Surface electromyography data is collected as data related to a motion during a certain exercise by the sensor 10. Thereafter, the motion evaluation device 30 performs a series of processing for extracting a section related to a motion desired to be evaluated from the surface electromyography data in the motion evaluation device 30, acquiring a feature amount, and evaluating the feature amount by the trained model 331, and an evaluation result for each motion is obtained as an output of the system. FIG. 8 shows a use case that it is also assumed that the evaluation results for the entire exercise including a plurality of motions are output by aggregating the evaluation results for the respective motions.

With the motion evaluation system 100 having the above-described configuration, it is possible to improve the evaluation accuracy of the motion of a person. Specifically, in the motion evaluation system 100, the noise removal unit 322 performs noise removal processing on the acquired time-series data, and the data processing unit 325 aligns the length of the data group for each on-set section while maintaining the time-series information, and performs down-sampling processing to compress the dimension of the data while holding the outline of the data. Thus, time distortion which is a weak point of similarity calculation due to Euclidean distance of the time-series data can be eliminated while leaving features of the waveform shape of data consisting of sequence between the time-series data and continuity of the on-set section, and a condition for fixing the number of samples required for calculation can be satisfied. Then, the motion evaluation device 30 evaluates the motion of the person on the basis of the compressed data of the on-set section. Therefore, it is possible to improve the evaluation accuracy of the motion of the person.

The data division unit 324 of the motion evaluation device 30 extracts a section from a start point to an end point as an on-set section on the time-series data. Thus, data of a section including the waveform of the motion of the person can be extracted from the time-series data after the noise is removed. Therefore, the influence of noise can be suppressed when estimating the motion of the person. Therefore, it is possible to improve the evaluation accuracy of the motion of the person.

The data division unit 324 of the motion evaluation device 30 compares a value of the time-series data with a value of a certain section immediately before, and extracts, as the on-set section, a section from the start point, which is a point at which the value of the time-series data increases or decreases by a threshold value or more, to the end point, which is a point that approaches an average value again at a time after the start point. In this way, when the value increases or decreases by a threshold value or more, it is assumed that some change has occurred with respect to the movement of the person. For example, it is assumed that the value of the time-series data has changed by a threshold value or more when a person starts any motion. Therefore, the data division unit 324 sets a point at which the value increases or decreases by a threshold value or more as a start point of the motion of the person. The data division unit 324 sets a point where the value is settled after the start point, that is, a point that approaches the average value as an end point where it is assumed that the motion of the person has been ended. In this way, the data division unit 324 can more strictly specify a section where it is assumed that the motion of the person has been performed. Therefore, it is possible to prevent the section including only noise from being included in the section for evaluating the motion of the person. Therefore, it is possible to improve the evaluation accuracy of the motion of the person.

Although the embodiments of the present invention have been described in detail with reference to the drawings, specific configurations are not limited to these embodiments, and designs and the like within a range that does not deviating from the gist of the present invention are also included.

### Industrial Applicability

The present invention can be applied to a technique for evaluating the motion of a person.

[Reference Signs List]

| | |
|---|---|
| 10, | 10-1 to 10-O Sensor |
| 20 | Sensor data acquisition device |
| 30 | Motion evaluation device |
| 40 | Learning device |
| 50 | Evaluation result receiving device |
| 31 | Communication unit |
| 32 | Control unit |
| 33 | Storage unit |
| 321 | Acquisition unit |
| 322 | Noise removal unit |
| 323 | Rectification unit |
| 324 | Data division unit |
| 325 | Data processing unit |
| 326 | Evaluation unit |
| 41 | Learning model storage unit |
| 42 | Training data input unit |
| 43 | Learning unit |

1. A motion evaluation method comprising:
removing noise in time-series data related to a motion of a person;
data of an on-set section in which the motion of the person is performed from the time-series data from which the noise has been removed;
aligning a length of the extracted data of the on-set section for each on-set section and compressing the data of the on-set section through down-sampling processing; and
evaluating the motion of the person on the basis of the compressed data of the on-set section.

2. The motion evaluation method according to claim 1, wherein, in the extracting, a section from a start point where the person is assumed to have started the motion to an end point where the person is assumed to have ended the motion is extracted as the on-set section on the time-series data.

3. The motion evaluation method according to claim 2, wherein, in the extracting, a value of the time-series data is compared with a value of a certain section immediately before and the on-set section is extracted from the time-series data with a point at which the value of the time-series data increases or decreases by a threshold value or more as the start point and a point that approaches an average value again at a time after the start point as the end point.

4. The motion evaluation method according to claim 2 3, wherein, in the aligning, with data of a plurality of the on-set sections extracted in the extracting, the length of the data of the on-set section is aligned for each on-set section by matching start points of the data of all the on-set sections with data having the earliest time at the start point and matching end points of the data of all the on-set sections with data having the latest time at the end point.

5. The motion evaluation method according to claim 1, wherein, in the evaluating, person is evaluated using a trained model trained to output an evaluation score by inputting the data of the on-set section.

6. A non-transitory computer readable storage medium that stores a computer program to be executed by the computer to perform:
removing noise in time-series data related to a motion of a person;
extracting data of an on-set section in which the motion of the person is performed from the time-series data from which the noise has been removed;
aligning a length of the extracted data of the on-set section for each on-set section and compressing the data of the on-set section through down-sampling processing; and
evaluating the motion of the person on the basis of the compressed data of the on-set section.

7. A motion evaluation system comprising:
a sensor configured to acquire time-series data related to a motion of a person;
a noise remover configured to remove noise in the time-series data;
an extractor configured to extract data of an on-set section in which the motion of the person is performed from the time-series data from which the noise has been removed;
a compressor configured to align a length of the extracted data of the on-set section for each on-set section and compress the data of the on-set section through down-sampling processing; and
an evaluator configured to evaluate the motion of the person on the basis of the compressed data of the on-set section.
